(19) United States
(12) Patent Application Publication
(10) Pub. No.: US 2009/0107863 A1
(43) Pub. Date: Apr. 30, 2009

(54) High-low adjustable lumber pad device

(76) Inventor: Fu-Hsing Tan, Lu Chu (TW)

Correspondence Address: TROXELL LAW OFFICE PLLC, Suite 1404, 5205 Leesburg Pike, Falls Church, VA, 22041, US

(21) Appl. No.: 11/976,601

(22) Filed: Oct. 25, 2007

(51) Int. Cl. A63B 55/00; A61F 5/00

(52) U.S. Cl. 206/315.3; 602/19

(57) ABSTRACT

A high-low adjustable lumber pad device for supporting a golf bag on the user's lower back is disclosed to includes a holder frame affixed to the golf bag and having a vertically extending coupling groove, and a lumber pad, which has a coupling bar vertically adjustably coupled to the coupling groove of the holder frame for supporting the holder frame and the golf bag on the lower part of the user's back stably.

### BACKGROUND OF THE INVENTION

1. Field of the Invention

The present invention relates to a lumber pad device for golf bag and more particularly, to a high-low adjustable lumber pad device that facilitates carrying of the golf bag.

2. Description of the Related Art

When playing the game of golf, every player may use a golf bag to carry a set of golf clubs. When carrying a golf bag on the shoulders, the golf clubs give a downward pressure to the shoulders. During walking, the golf bag may oscillate, and the person carrying the golf bag may feel uncomfortable. Further, when the golf bag is oscillating, the storage golf clubs may be forced to hit or rub against one another, causing damage.

### SUMMARY OF THE INVENTION

The present invention has been accomplished under the circumstances in view. It is the main object of the present invention to provide a high-low adjustable lumber pad device, which supports a golf bag stably on the lower part of the back of the person who carries the golf bag on the shoulders. To achieve this and other objects of the present invention, the high-low adjustable lumber pad device is comprised of a holder frame affixed to a golf bag, and a lumber pad for supporting the holder frame on the lower part of the back of a person. The holder frame has a vertically extending coupling groove. The lumber pad has a coupling bar disposed at the back side and vertically adjustably coupled to the coupling groove of the holder frame.

### BRIEF DESCRIPTION OF THE DRAWINGS

FIG. 1 is an oblique front elevation of a high-low adjustable lumber pad device in accordance with the present invention.

FIG. 2 is an oblique rear elevation of the high-low adjustable lumber pad device in accordance with the present invention.

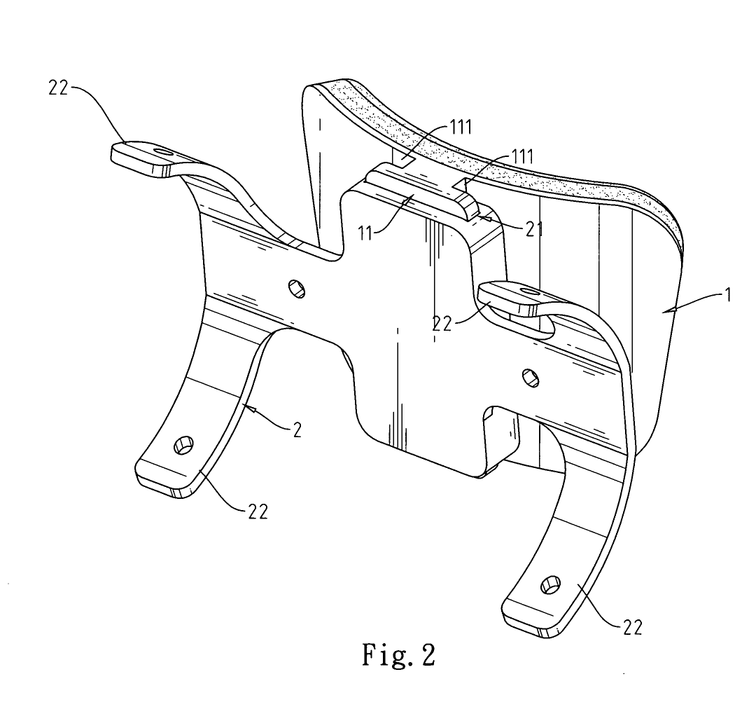

FIG. 3 is an exploded view of the high-low adjustable lumber pad device in accordance with the present invention.

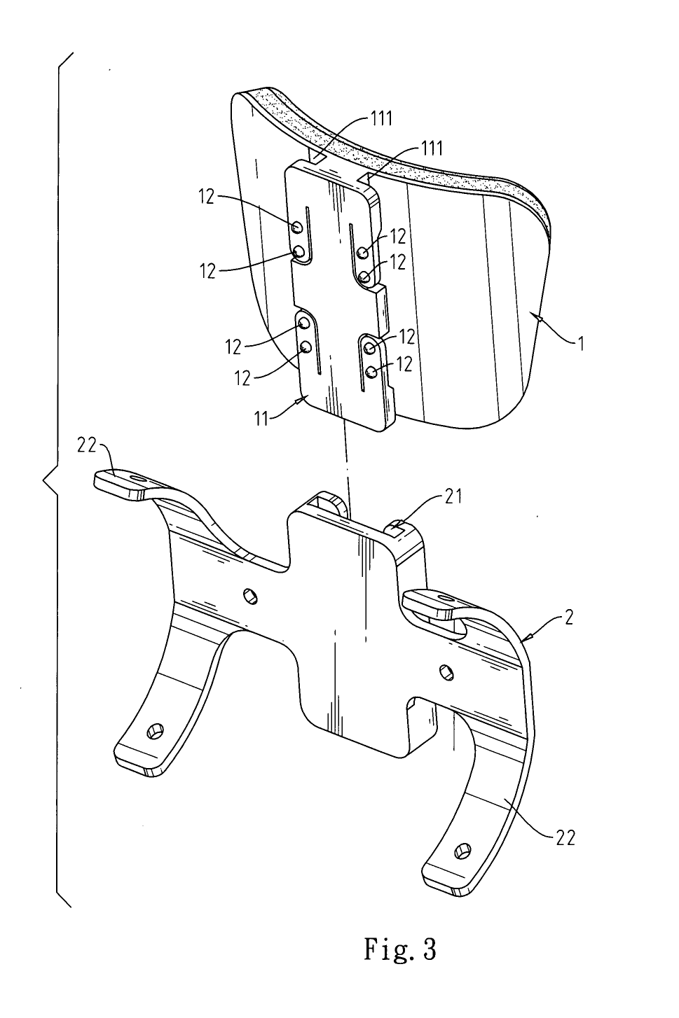

FIG. 4 is a sectional elevation of a part of the holder frame of the high-low adjustable lumber pad device in accordance with the present invention.

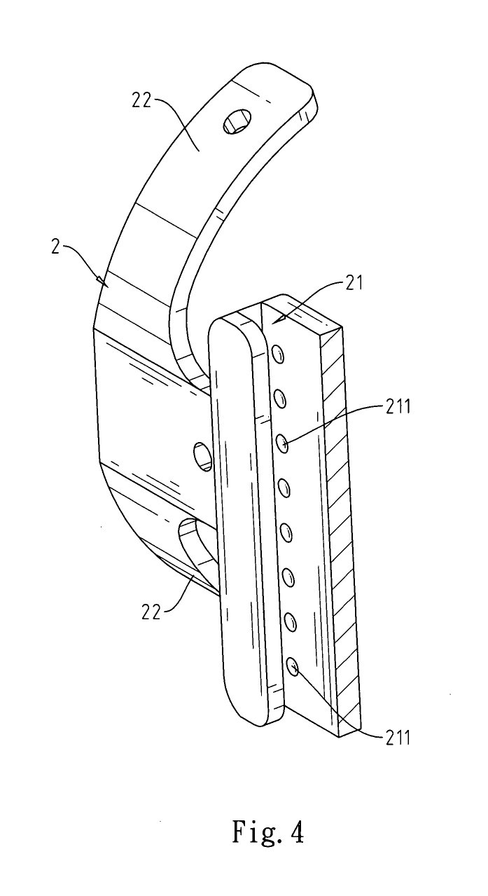

FIG. 5 is a schematic drawing of the high-low adjustable lumber pad device in accordance with the present invention, showing adjustment of the elevation of the lumber pad relative to the holder frame.

FIG. 6 is a schematic drawing showing the high-low adjustable lumber pad device of the present invention fastened to a golf bag.

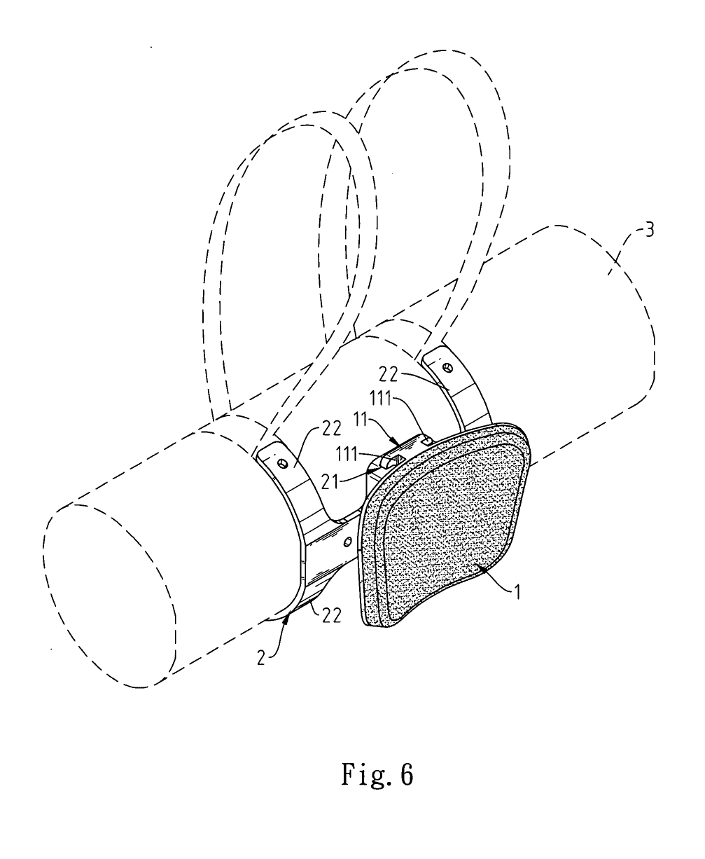

FIG. 7 is a schematic drawing showing a status of use of the present invention.

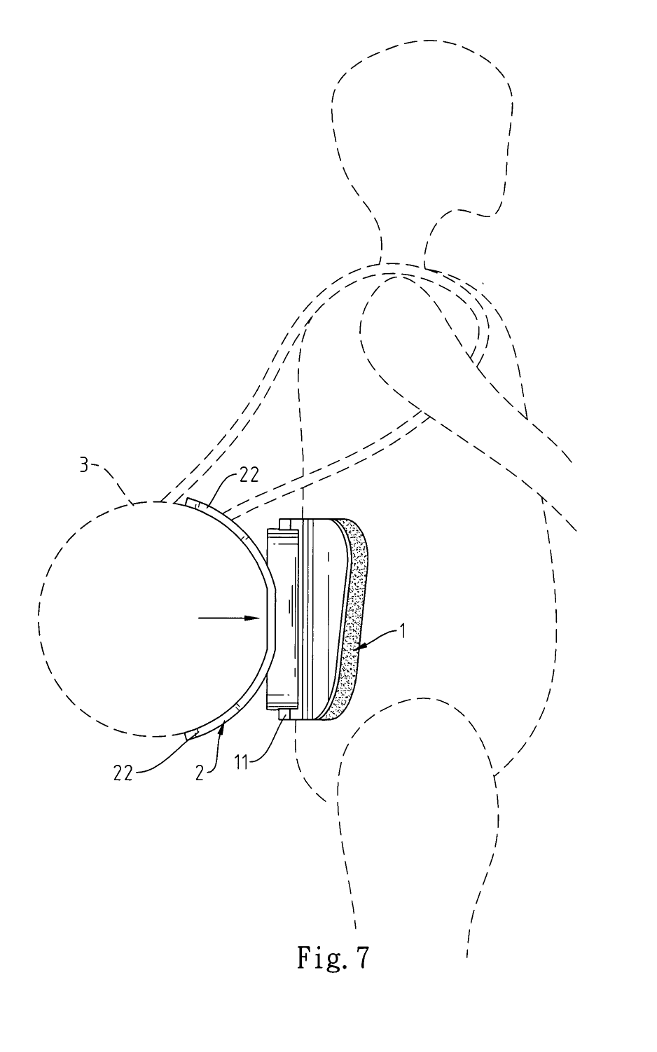

### DETAILED DESCRIPTION OF THE PREFERRED EMBODIMENT

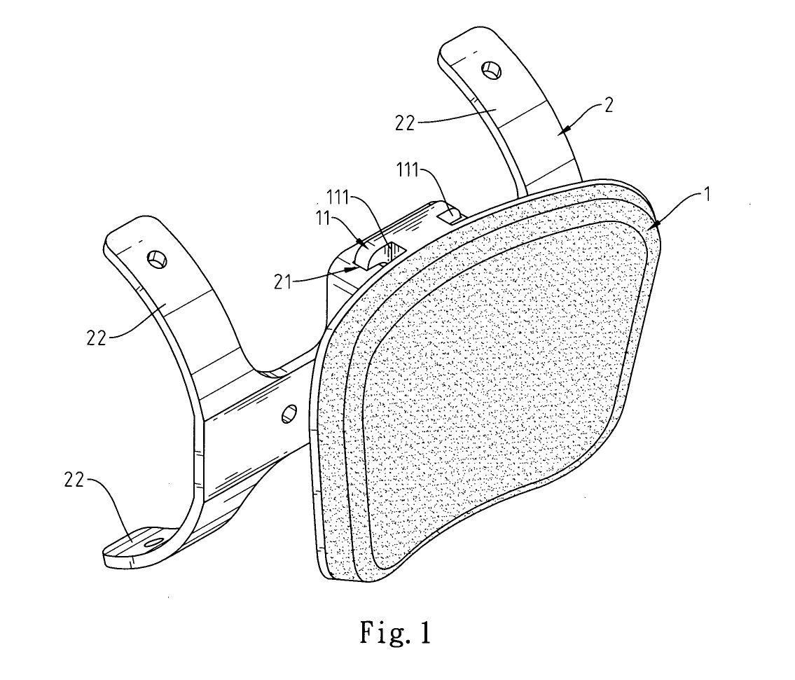

Referring to FIGS. 1˜4, a high-low adjustable lumber pad device in accordance with the present invention is shown comprised of a lumber pad 1 and a holder frame 2.

The lumber pad 1 is orthopedically engineered to fit the lumber curve. The lumber pad 1 comprises a T-shaped coupling bar 11 protruded from its back wall. The T-shaped coupling bar 11 defines with the back wall of the lumber pad 1 two coupling grooves 111. Further, the T-shaped coupling bar 11 comprises a plurality of elastic raised positioning portions 12 arranged in two lines along the length of the T-shaped coupling bar 11.

The holder frame 2 comprises a coupling groove 21 that fits the cross section of the T-shaped coupling bar 11 so that the T-shaped coupling bar 11 can be coupled to and moved along the coupling groove 21, and a plurality of recessed positioning portions 211 disposed inside the coupling groove 21 corresponding to the raised positioning portions 12 of the T-shaped coupling bar 11. The holder frame 2 further comprises two mounting bars 22 symmetrically disposed at two opposite sides relative to the coupling groove 21.

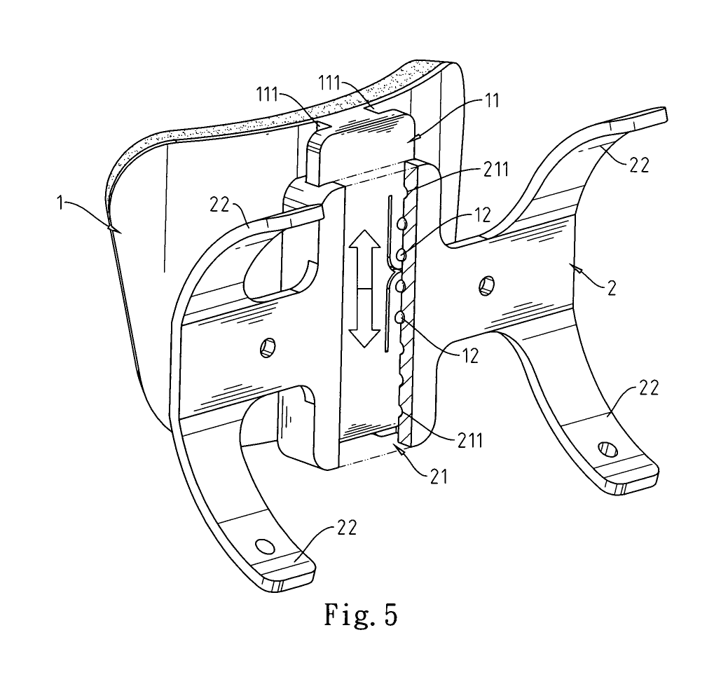

Referring to FIGS. 5˜7, the two mounting bars 22 of the holder frame 2 are affixed to the periphery of a golf bag 3 at a suitable location. Before use, the lumber pad 1 is pulled or pushed to move the T-shaped coupling bar 11 along the coupling groove 21 of the holder frame, changing the engagement order between the raised positioning portions 12 of the T-shaped coupling bar 11 and the recessed positioning portions 211 of the holder frame 2, adjusting the elevation of the lumber pad 1 relative to the holder frame 2. When the user carries the golf bag 3 on the users shoulders, the lumber pad 1 is attached to the lower back of the user's body. Thus, the golf bag 3 is stably supported on the user's back.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A high-low adjustable lumber pad device comprising:
a holder frame, said holder frame comprising a vertically extending coupling groove, and two mounting bars symmetrically disposed at two opposite sides relative to said coupling groove for fastening to a golf bag;
a lumber pad for supporting said holder frame on the back of a person, said lumber pad comprising a coupling bar vertically disposed at a back side thereof slidably coupled to said coupling groove of said holder frame; and
lock means to lock said coupling bar of said lumber pad to said coupling groove of said holder frame.

2. The high-low adjustable lumber pad device as claimed in claim 1, wherein said lumber pad comprises two coupling grooves disposed at two sides of said coupling bar for enabling said coupling bar to be coupled to the coupling groove of said holder frame.

3. The high-low adjustable lumber pad device as claimed in claim 1, wherein said lock means comprises a plurality of elastic raised positioning portions protruded from said coupling bar of said lumber pad, and a plurality of recessed positioning portions disposed in the coupling groove of said holder frame for receiving said elastic raised positioning portions.
